(19) United States
(12) Patent Application Publication
(10) Pub. No.: US 2016/0177250 A1
(43) Pub. Date: Jun. 23, 2016

(54) TISSUE TRANSFER SYSTEM

(71) Applicant: CYTORI THERAPEUTICS, INC., San Diego, CA (US)

(72) Inventors: Douglas M. Arm, Carlsbad, CA (US); John Rodriguez, Vista, CA (US); David G. Matsuura, Del Mar, CA (US); Philip Simpson, Escondido, CA (US); Raphael Shapiro, La Jolla, CA (US); Min Zhu, San Diego, CA (US)

(21) Appl. No.: 14/750,932

(22) Filed: Jun. 25, 2015

Related U.S. Application Data
(60) Provisional application No. 62/017,117, filed on Jun. 25, 2014.
(60) Provisional application No. 62/075,762, filed on Nov. 5, 2014.

(51) Int. Cl. C12M 1/26; C12M 1/12; C12M 1/00

(52) U.S. Cl. CPC C12M 33/04; C12M 23/26; C12M 37/00; C12M 33/12; C12M 37/04

(57) ABSTRACT

Described herein are devices for the transfer of tissue into tissue processing devices. In some embodiments, the devices provide for the transfer of tissue from a tissue collection vessel to a tissue processing vessel. In some embodiments, the devices and methods provide for direct harvest of the tissue into a tissue processing vessel. In some embodiments, the devices and methods provide for transfer of harvested tissue within a tissue collection container to a tissue processing container.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit of priority to U.S. Provisional Patent Application No. 62/017,117, filed on Jun. 25, 2014, and U.S. Provisional Patent Application No. 62/075,762, filed on Nov. 5, 2014, the contents of both applications are hereby expressly incorporated by reference in their entireties.

### BACKGROUND

Since the discovery of adult stem cells and other cell types with therapeutic benefit in various human tissues (e.g., adipose, bone marrow, cord blood, heart, liver, skin, etc.), several methods and devices have been used to harvest tissue and process tissue to isolate certain cell populations therefrom. It is often desirable to use isolated populations of cells or tissue in a clinical setting and/or a research setting. As such, methods and devices used for tissue harvest and any downstream processing ideally preserve harvest efficiency, cell viability, cell output, and sterility. In addition, particularly in clinical settings, the speed and efficiency of transfer of harvested tissue to a processing device to isolate desired cells is important. It is also desirable to reduce or eliminate steps requiring human manipulation in order to simplify the steps of tissue harvest and processing and to minimize operator error and inter-operator variability.

Accordingly, the need for devices that simplify the process and improve the handling and sterility of tissue harvest and processing is manifest.

### SUMMARY

Embodiments disclosed herein relate to devices useful for the transfer of tissue into a tissue processing device, methods of transferring tissue to tissue processing devices using the devices described herein, and kits for transferring tissue to tissue processing devices.

### BRIEF DESCRIPTION OF THE DRAWINGS

FIG. 1 is an illustration of an exemplary tissue transfer device utilizing negative pressure to facilitate tissue transfer as described herein.

FIG. 2 is an illustration of an exemplary tissue transfer device utilizing negative pressure to facilitate tissue transfer as described herein.

FIG. 3A is a photograph of an exemplary tissue transfer device utilizing negative pressure to facilitate tissue transfer as described herein. Shown is a book-shaped canister, in an open position, providing access to the interior chamber of the canister.

FIG. 3B is a photograph of the tissue transfer device shown in FIG. 3A, including a tissue vessel disposed therein.

FIG. 3C is a photograph of the tissue transfer device shown in FIG. 3A, in a closed position.

FIG. 4 is an illustration of an exemplary tissue transfer device as described herein.

FIG. 5 is an illustration of an exemplary tissue transfer device as described herein.

FIG. 6 is an illustration of an exemplary tissue transfer device as described herein.

FIG. 7 is an illustration of an exemplary tissue transfer device as described herein.

FIG. 8 is an illustration of an exemplary tissue transfer device as described herein.

### DETAILED DESCRIPTION

The embodiments described herein relate to devices and methods of their use to facilitate transfer of tissue to a processing device, where tissue can be processed, e.g., in order to liberate and/or isolate and/or concentrate desired cell populations therefrom. Advantageously, some embodiments described herein are configured to allow or enable the direct transfer, e.g., from a tissue harvesting apparatus such as a vacuum assisted cannula, or the like, into a tissue processing device, such as a tissue processing bag, or other processing device, as described in further detail below. Other embodiments are advantageously designed to allow or enable the transfer of tissue from a tissue collection device (e.g., a vessel into which tissue has been harvested), into a tissue processing device, such as a tissue processing bag, or the like, as described in further detail below. The devices disclosed herein advantageously maintain a functionally closed system.

As used herein, the term “tissue” includes any tissue harvested from a subject, such as a mammal (e.g., human, ape, monkey, horse, dog, cat, mouse, rat, rabbit, pig, or other mammal). Non-limiting examples of tissues include those tissues which include regenerative cells, such as bone marrow, placenta, adipose tissue, skin, eschar tissue, endometrial tissue, adult muscle, corneal stroma, dental pulp, Wharton's jelly, amniotic fluid, brain, muscle, fascia, umbilical cord and the like. The skilled person will readily appreciate that the embodiments described herein can be used in the context of harvest and downstream processing of tissues such as plasma, muscle, fascia, organs (pancreas, lung, kidney, liver, skin, heart, eye, brain, CNS, and the like), and the like.

As used herein, the term “closed fluid/tissue pathway” or “closed system” refers to a system in which material therein is not exposed to the external environment. The term “functionally closed,” as used herein, refers to a system that totally closed at the point of manufacture except for preconnected solutions and/or preconnected FDA approved bacteriostatic filters on spike and port connections.

### Negative Pressure-Based Tissue Transfer

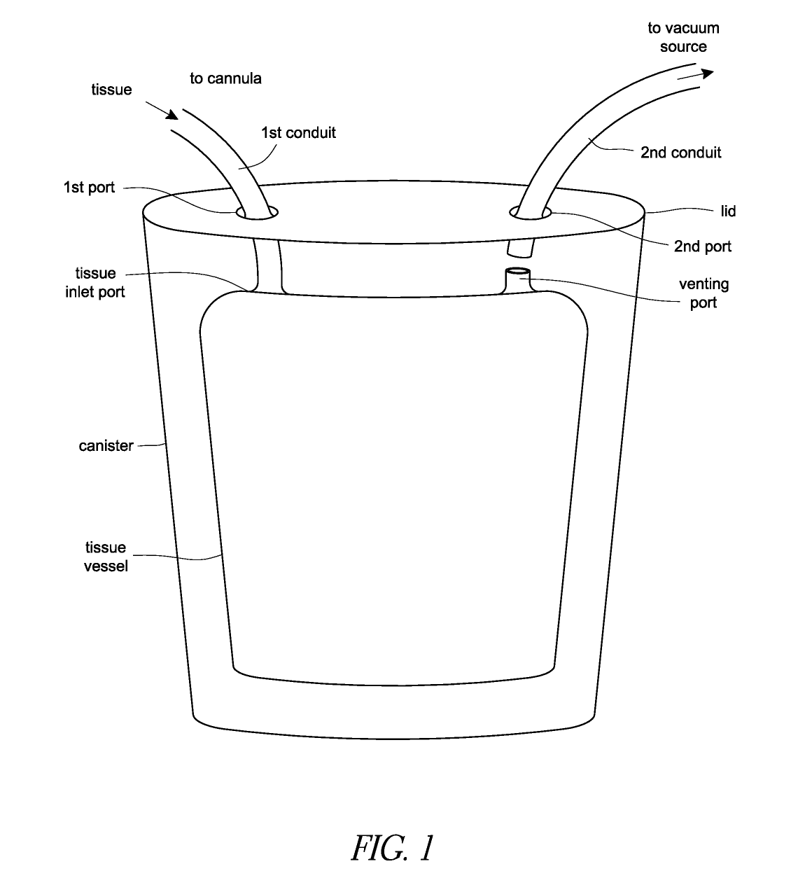

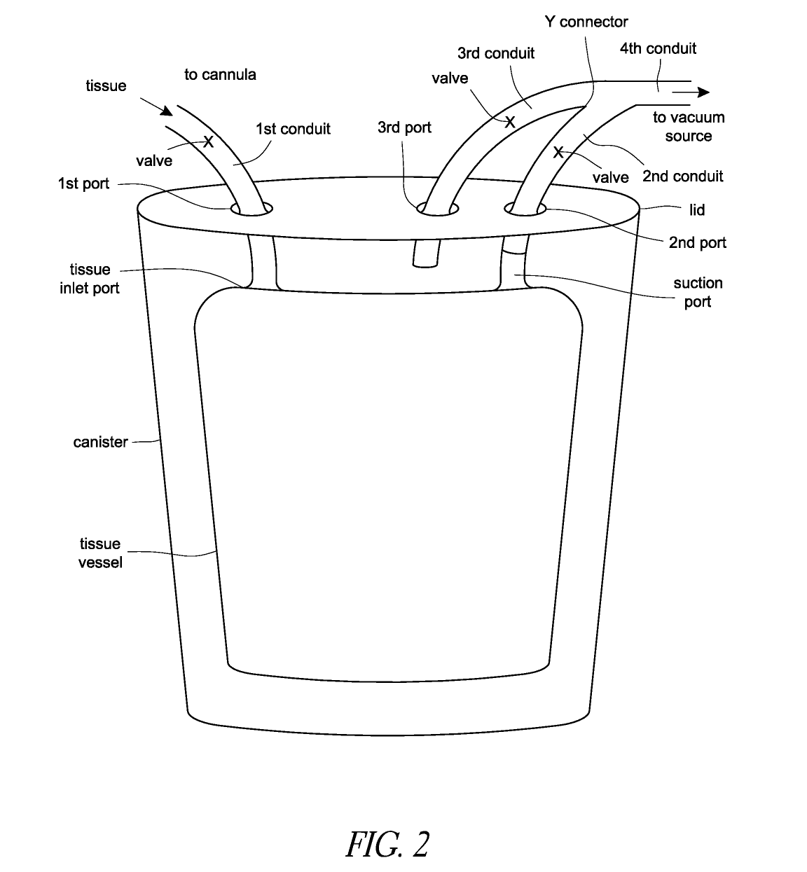

Some of the embodiments disclosed herein relate to devices configured to facilitate tissue transfer using negative pressure, i.e., a vacuum. FIGS. 1 and 2 illustrate embodiments useful for the direct transfer of tissue into a tissue processing device using negative pressure.

In the embodiments shown in FIGS. 1 and 2, harvested tissue can be transferred directly from the subject into a tissue vessel, e.g., a tissue processing device for downstream manipulation of harvested tissue, or tissue vessel used for other purposes. The skilled artisan will readily appreciate that the embodiments illustrated in FIGS. 1 and 2 advantageously allow for harvested tissue to be transferred directly into a flexible tissue vessel, although the skilled person will appreciate that these embodiments could just as readily be utilized for direct transfer to a rigid tissue vessel. Specifically, the embodiments disclosed herein are uniquely and advantageously configured to allow for the use of a vacuum or negative pressure to transfer tissue directly into a flexible bag, while minimizing or preventing implosion and/or structural compromise of the flexible bag during tissue transfer. The embodiments shown in FIGS. 1 and 2 advantageously minimize or eliminate the pressure differential in the interior of the tissue vessel (e.g., flexible bag) and the exterior of the tissue vessel (flexible bag). In other words, the embodiments shown in FIGS. 1 and 2 balance the pressure within the inner vessel chamber of the tissue vessel, and outside (i.e., exterior) of the tissue vessel.

The tissue transfer device depicted in FIGS. 1 and 2 includes a canister that houses the tissue vessel therein. As such, the canister can have an interior chamber and an exterior. As described in further detail below, some embodiments disclosed herein advantageously balance, or substantially balance, the pressure within the inner chamber of a tissue vessel (including, but not limited to, a tissue processing bag, or the like, as described in further detail below), and the interior chamber of the canister. Accordingly, the canister is made of material capable of withstanding negative pressure without imploding, or compromising the canister structure. Specifically, the canister is desirably rigid and manufactured from a material such as polycarbonate, acrylic, ABS, ethylene vinyl acetate or styrene-butadiene copolymers (SBC) or the like. In preferred embodiments, the canister is manufactured from medical grade materials. Preferably, the canister is manufactured from material that can be sterilized, e.g., using dry heat, steam, UV light, chemicals, or the like, such that the canister is re-usable. In some embodiments, the canister is disposable. Preferably, the canister is manufactured from material that is bisphenol-A (BPA)-free, and/or pyorgen-free, or the like. The rigid plastics of the canisters, lid, and tubes may be formed using conventional blow or injection molding techniques, extrusion, or other suitable plastic shaping and forming techniques. In some embodiments, the canister body can have a capacity of 10 ml to 10 L, or any volume in between.

In the embodiments illustrated in FIGS. 1 and 2, the canisters include a body and a lid. The lid is releasably engageable with the canister body. In some embodiments, the lid can snap into place to form a seal with the canister body, e.g., via complementary flanges on the canister body (i.e., on the rim of the canister body) and the lid. In some embodiments, the lid can slide into place, e.g., to fit within an opening on the canister body. For example, in some embodiments, the body of the canister has a bottom, sidewalls, and a top with an opening therein that provides access to the interior chamber of the canister. The lid can thus be a complementary shape to, and forms a seal when inserted into, the opening in the top of the canister body. In some embodiments, the canister and lid can be opened and closed, i.e., sealed against the canister body via a hinging type mechanism. The skilled person will readily appreciate that the body and lid can have numerous different configurations. Regardless of the configuration of the canister lid and body, the lid and body together fit together to form a canister capable of maintaining a pressure differential between the interior chamber of the canister and the external environment. As explained in further detail below, in some embodiments, e.g., the exemplary embodiments illustrated in FIGS. 1 and 2, the assembled canister (i.e., the body and lid together), are capable of maintaining a negative pressure, e.g., between about 0.05 kPa and 100 kPa or between about 0.015 inches Hg and 29 inches Hg, within the internal chamber, without compromising the canister structure.

In the embodiment shown in FIGS. 1 and 2 the canister has at least a first port and a second port. The ports have an exterior end located on the exterior of the canister, and an interior end located on the interior chamber. In the embodiments shown in FIGS. 1 and 2, the ports are located on the lid, although one or both of the first and second ports can also be located on the canister body. The exterior end of the first port is configured for sterile attachment (either directly or via a sterile connector), to a first conduit, such as a flexible tubing or the like, configured for the sterile transfer of tissue, liquid, and other material therethrough. The tubing and conduits described herein are preferably constructed from rigid or flexible material capable of withstanding the negative pressures introduced into the system without collapsing. The conduits are also preferably constructed of a transparent or translucent material to facilitate observation material passing therethough by the device operator. The first conduit can be connected to an instrument, e.g., a cannula, or the like, for suction-assisted tissue harvest, thereby allowing for a sterile fluid/tissue pathway between the tissue harvest instrument and the first port leading into the interior chamber of the canister. As such, as mentioned above, the embodiments described herein can provide a functionally closed pathway between the tissue harvesting device and the tissue vessel.

The exterior end of the second port is configured for attachment to a second conduit, providing a pathway to a vacuum source. The skilled person will readily appreciate that both manual and motorized vacuum sources can be used in the embodiments disclosed herein (e.g., vacuum pumps, syringes, and the like). Many different conventional, commercially available vacuum sources are useful in the embodiments disclosed herein. By way of example only, a vacuum source such as vacuums available from MD Resource (Danville, Calif.), vacuum sources such as the HERCULES™, WHISPERATOR™, and other general aspirators available from Wells-Johnson (Tucson, Ariz.), and having a typical operating range from 1-30 inches Hg, are useful in the embodiments described herein. With the vacuum source activated, a vacuum pressure relative to the ambient pressure will be generated and maintained in interior chamber of the canister (and/or tissue vessel, as described in further detail herein below).

A tissue vessel can be disposed within the interior chamber of the canister. In preferred embodiments, the tissue vessel is flexible, e.g., a flexible bag such as those described in U.S. Patent Application Publication No. 2010/0279405. Accordingly, in some embodiments, the tissue vessel is a flexible, collapsible bag, that has an interior vessel chamber, a tissue inlet port that is configured for ingress and egress of material (e.g., tissue, fluids, and the like), into an out of the interior vessel chamber, and which is also configured for sterile connection to the interior end of the first port on the canister. The tissue vessel can also include one or more venting ports that each enable sterile ingress and egress of material into and out of the interior vessel chamber, so as to maintain a functionally closed system within the tissue vessel.

Exemplary tissue vessels useful in the embodiments disclosed herein include, for example, flexible tissue vessels such as those described in U.S. Patent Application Publication No. U.S. 2010/0279405, the disclosure regarding a flexible tissue container is herein incorporated by reference. Other non-limiting examples of tissue vessels useful in the embodiments disclosed herein include, but are not limited to, those described in U.S. Pat. Nos. 7,390,484, 7,585,670, 7,687,059, 8,309,342, 8,440,440, U.S. Patent Application Publication No's. 2013/0164731, 2013/0012921, 2012/0164113, 2008/0014181. 2013/180952, 2013/0324966, 2010/0285521, 2013/0034524 International Patent Application Publication No. WO 2009/073724, WO 2013/030761, WO 2014/03969, WO 2013/106655, WO 2014/036094, and the like, each of which is herein incorporated by reference. In some embodiments, the tissue vessel has a capacity of at least 25 ml, 30 ml, 40 ml, 50 ml, 60 ml, 70 ml, 80 ml, 90 ml, 100 ml, 125 ml, 150 ml, 175 ml, 200 ml, 225 ml, 250 ml, 275 ml, 300 ml, 325 ml, 350 ml, 375 ml, 400 ml, 425 ml, 450 ml, 475 ml, 500 ml, 525 ml, 550 ml, 575 ml, 600 ml, 625 ml, 650 ml, 675 ml, 700 ml, 725 ml, 750 ml, 775 ml, 800 ml, 825 ml, 850 ml, 875 ml, 900 ml, 925 ml, 950 ml, 975 ml, 1 L, 1.25 L, 1.5 L, 1.75 L, 2 L, 2.25 L, 2.5 L, 2.75 L, 3 L, 3.25 L, 3.5 L, 3.75 L, 4 L, 4.25 L, 4.5 L, 4.75 L, 5 L, 5.25 L, 5.5 L, 5.75 L, 6 L, or greater, or any volume in between.

In the embodiment shown in FIG. 1, the exterior end of the first port is configured for sterile connection to a tissue harvest device, and for providing a sterile pathway for passage of tissue from a harvest site to the interior chamber of the tissue vessel. In the embodiment shown in FIG. 1, the tissue vessel includes a venting port that allows for the sterile ingress and egress of material in and out of the interior chamber of the tissue vessel. In the embodiment shown in FIG. 1, the venting port of the tissue vessel is not connected to the tissue canister, and provides for the flow of gases between the interior chamber of the canister and the interior chamber of the tissue vessel. As such, the venting port of the tissue vessel can function to balance the pressure, or minimize pressure differential, between the interior chamber of the canister and the interior chamber of the tissue vessel. Although the particular embodiment shown in FIG. 1 includes one venting port, the skilled person will immediately appreciate that the tissue vessel can include more than one, i.e., 2, 3, 4, 5, 6, 7, 8, 9, 10, or more, venting ports. Preferably, the size and number of the venting ports of the tissue vessel are such that the pressure in the interior vessel chamber and the exterior of the tissue vessel is substantially equal (or wherein the differential is such that tissue vessel is not structurally compromised), when a vacuum source connected to the second port of the canister is activated thereby generating negative pressure in the interior chamber of the canister.

As used herein, the term “substantially equal,” i.e., in connection with the pressure in the interior chamber of the tissue vessel and the exterior of the tissue vessel, refers to a differential of less than 10 inch Hg, less than 9 inch Hg, less than 8 inch Hg, less than 7 inch Hg, less than 6 inch Hg, less than 5 inch Hg, less than 4 inch Hg, less than 3 inch Hg, less than 2 inch Hg, less than 1 inch Hg, less than 0.9, inch Hg, less than 0.8 m inch m Hg, less than 0.7 inch Hg, less than 0.6 inch Hg, less than 0.5 inch Hg, less than 0.4 inch Hg, less than 0.3 inch Hg, less than 0.2 inch Hg, less than 0.1 inch Hg, or less, or any amount in between. Although the embodiment shown in FIG. 1 depicts one venting port, the skilled person will readily appreciate that the tissue vessel can include 2, 3, 4, 5, 6, 7, 8, 9, 10, or more venting ports. Preferably, the venting ports include a filter therein, e.g., 0.2 μm or less, such that material entering into the interior chamber of the tissue vessel (e.g., air), is sterile, so as to maintain a functionally closed system.

In some embodiments, the interior end of the tissue inlet port of the tissue vessel includes a straw-like structure that extends downwards into the interior chamber of the tissue vessel, such that tissue does not enter the interior chamber of the tissue vessel in close proximity to the venting port. The straw includes a lumen through configured for the passage of material (e.g., tissue), therethrough. The bottom end of the straw extends into the interior chamber, while the top end of the straw is connected to the tissue inlet port. The bottom end of the straw can extend a distance downwards into the tissue vessel that is at least about a quarter of the height of the tissue vessel, in order to advantageously facilitate filling of the tissue vessel, i.e., transfer of tissue into the interior chamber of the tissue vessel.

In some embodiments, the tissue vessel is a flexible, collapsible bag that includes a mesh that defines a first and second chamber of the interior vessel chamber. The mesh can have a plurality of pores that pass liquids, tumescent fluids, red blood cells, and wash solutions and retain cellular material such as mature adipocytes, regenerative cells (e.g., stem cells, progenitor cells, precursor cells, and the like), and connective tissue. The tissue vessel can also include a screen within the second chamber that creates a space between the mesh and the flexible, collapsible bag, and which wicks tumescent fluids, red blood cells, and wash solutions from the first chamber into the second chamber. The tissue inlet port provides access for aseptic introduction of tissue into the first chamber. The one or more venting ports are configured to allow for sterile ingress and egress of material into and out of the first chamber and/or the second chamber. In some embodiments, the flexible, collapsible bag includes a drain port configured for the aseptic removal of liquids, tumescent fluids, red blood cells, and wash solutions from the second chamber. In some embodiments, the flexible, collapsible bag includes an auxiliary port configured to provide for sterile ingress and egress of material in and out of the first chamber. In some embodiments, the devices include valves that function to block the movement of material through the first or the second conduit when in a closed position, and that allow the movement of material through the conduits when in an open position. In some embodiments, one or more of the venting ports, auxiliary ports, or drain ports, can include a valve that blocks the passage of material through the port when in a closed position, and allows the passage of material through the port when in an open position.

Turning to the embodiment shown in FIG. 2, the canister has at least three ports, i.e., a first port (tissue), a second (vacuum) port and a third (equalizing) port. The exterior end of the first port is configured for attachment a tissue harvesting instrument. In some embodiments, the first port is connected to the tissue harvesting instrument via a conduit, (i.e., first conduit), that is connected at the other end to the harvesting instrument, e.g., a cannula, or the like, for suction-assisted tissue harvest, thereby allowing for a sterile fluid/tissue pathway between the tissue harvest instrument and the first port leading into the interior chamber of the canister. The second and third port can be coupled to a vacuum manifold that includes a second conduit and a third conduit, that are coupled to the second (vacuum) and third (equalizing) port, respectively, on the canister. The second and third conduits are both coupled (e.g., via a Y-connector to a single, fourth conduit, or other configuration), to the vacuum source.

In some embodiments, the first conduit, the second conduit, and the third conduit (or any combination thereof) can include a valve. In the open position, the valve enables transfer of material through the conduit, and in the closed position, the valve blocks transfer of material through the conduit. In some embodiments, the valve can be a pinch valve, e.g., that is placed over the exterior of the conduit. The skilled person will readily appreciate, however, that any valve known to those in the art, or discovered in the future, are appropriate in the embodiments disclosed herein, including but not limited to pneumatic valves, hydraulic valves, motor valves, and the like.

In the embodiment shown in FIG. 2, the tissue vessel disposed within the canister can be a flexible tissue vessel that has an interior vessel chamber, and a tissue inlet port that is configured for sterile connection to the interior end of the first port of the canister (e.g., directly, or via a sterile connection device). The tissue vessel also includes a suction port that enables ingress and egress of material in and out of the interior vessel chamber. As shown in FIG. 2, the suction port is configured for sterile connection to the interior end of the second port of the canister.

In the embodiment shown in FIG. 2, the first port of the canister is connected to the tissue inlet port of the tissue vessel, and the vacuum port of the canister is connected to the suction port of the tissue vessel. The equalizing port of the canister is not connected to the tissue vessel. To transfer tissue into the tissue vessel, the first, second and third conduits are open—that is, in embodiments wherein any of the first, second or third conduits include a valve, the valve(s) is in the open position. In some embodiments, the size of the third (equalizing) port and third conduit is such that the pressure in the interior vessel chamber and the exterior of the tissue vessel is substantially equal (or is balanced such that the pressure differential between the interior chamber of the tissue vessel and the exterior of the tissue vessel is not great enough to structurally compromise the tissue vessel), when a vacuum source connected to the second port of the canister is activated, thereby generating negative pressure in the interior chamber of the canister. In some embodiments, the pressure in the interior vessel chamber and the exterior of the tissue vessel/interior chamber of the canister is balanced by adjusting the diameter of the conduits, e.g., via adjustable pinch valves or the like, attached to the exterior of the first, second, and or third conduits. In some embodiments, the diameter of the conduits, e.g., from the first, second and third ports of the canister are the same. In some embodiments, the diameters of the conduits (i.e., their interior lumens) are not equal. For example, in some embodiments, the diameter of the lumen of the conduits leading from the second and third ports of the canister can be different. For example, the diameter of the suction port, the second (vacuum) port of the canister, and/or the second conduit can be smaller than the diameter of the third (equalizing) port of the canister and/or the third conduit.

In some embodiments, the tissue vessel is a flexible, collapsible bag that has an interior chamber with a mesh disposed therein that defines a first chamber and a second chamber of the interior chamber and a screen, as described above. In some embodiments, the suction port provides sterile ingress and egress of material in and out of the interior of the first chamber. In some embodiments, the suction port provides sterile ingress and egress of material in an out of the interior of the second chamber. In some embodiments, the tissue vessel can also include one or more venting ports, auxiliary ports, and drain ports, as described above. In some embodiments, the tissue vessel includes a straw extending downwards into the interior chamber (i.e., into the first chamber) of the tissue vessel from the tissue inlet port.

In some embodiments, vacuum pressure can be used to remove material from the second chamber of the tissue vessel. For example, in the embodiment depicted in FIG. 2, the first conduit and the third conduit can include valves. The suction port of the tissue vessel can be a drain port that provide for the ingress and egress of material in and out of the second chamber, e.g., the chamber into which liquids, tumescent fluids, red blood cells, and wash solutions are wicked following vacuum assisted harvest of adipose tissue. Following tissue harvest, valves for the first and third conduits are closed, and a vacuum source connected to the manifold is activated, thereby causing material in the second chamber of the tissue vessel to be extracted through the suction/drain port. In some embodiments, the device can include a waste canister that is in line with the conduit leading from the drain port and to the vacuum source, such that material in the second chamber of the tissue vessel is collected in the waste canister, and does not enter the vacuum source.

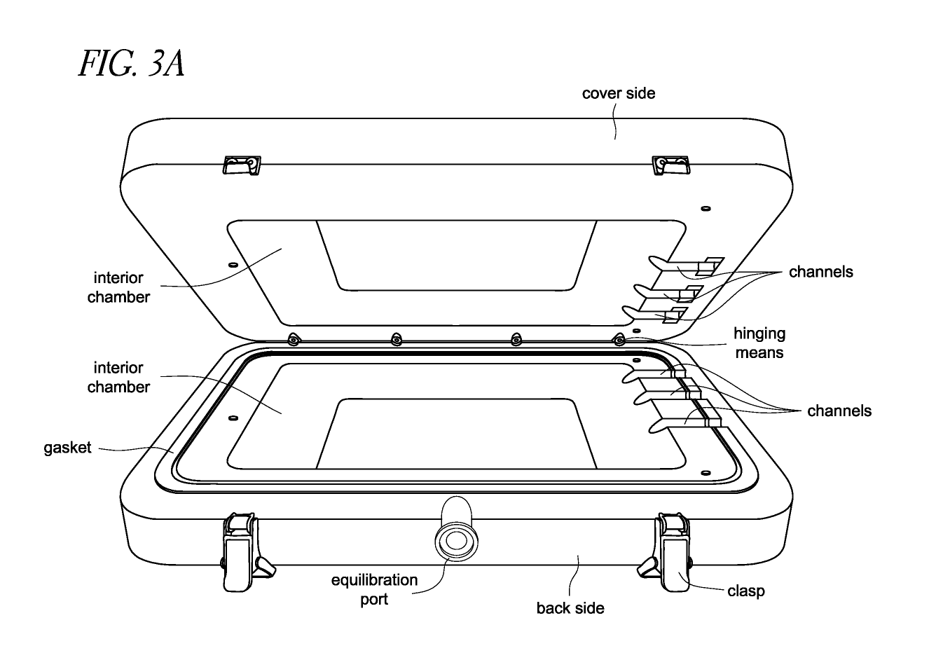

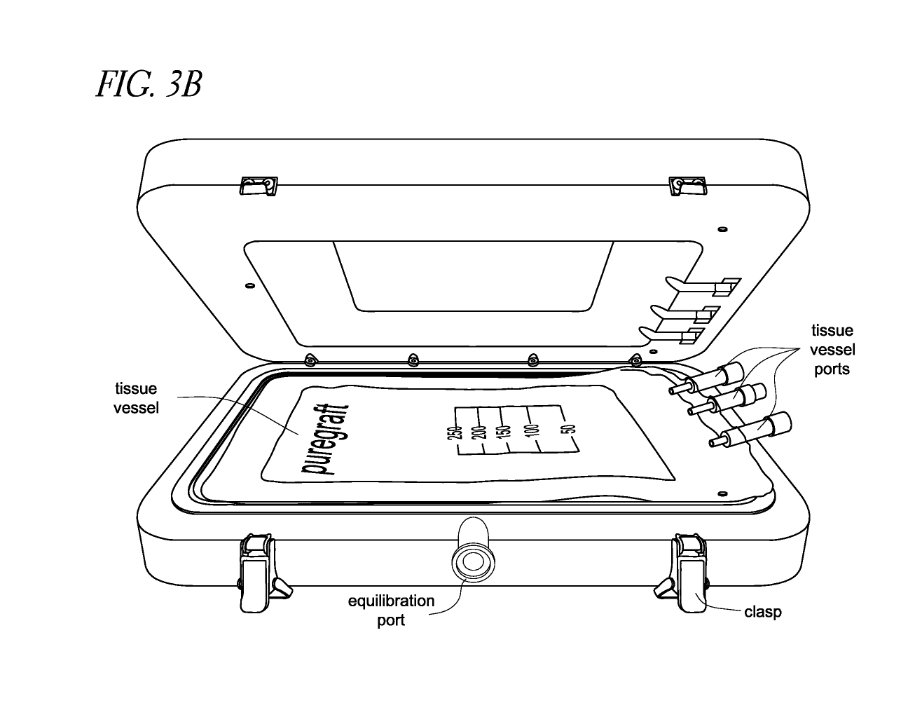

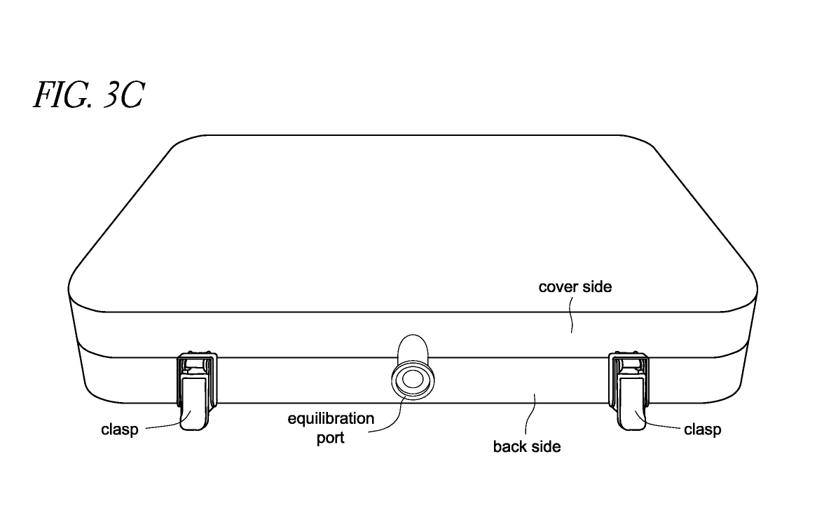

Turning to the embodiment shown in FIGS. 3A-3C, various embodiments provide a tissue canister that is book shaped, and that is configured to house a tissue vessel having an interior chamber configured to receive tissue, and at least one port, i.e., a tissue inlet port, configured for the sterile ingress and egress of material in to and out of the interior chamber of the tissue vessel, as described herein above. The book-shaped canister can have a cover side and a back side that are movably connected to each other, e.g., via a hinging means. FIG. 3A illustrates a book-shaped tissue canister as provided herein in an open position. In the open position, the book shaped canister provides access to the interior chamber configured to house a tissue vessel (e.g., a flexible tissue vessel as described elsewhere herein). In a closed position, the interior chamber of the book shaped canister forms a seal around the edges of the canister, while accommodating ports present on a tissue vessel housed within the interior chamber of the canister. For example, in the embodiment shown in FIG. 3A, the book-shaped canister includes a gasket that runs along the contours of the back side, and that forms a seal between the back side and the cover side when the canister is in a closed position. FIG. 3B illustrates a tissue vessel disposed within a book-shaped canister as described herein. While the embodiment illustrated in FIG. 3B shows a tissue vessel with three ports, it is understood that the tissue vessel can include 1, 2, 3, 4, 5, 6, 7, 8, 9, 10 or more ports, that provide access into an internal chamber of the tissue vessel. The cover side and/or the back side have one or more channels disposed therein that are configured to accommodate the structure of one or more respective ports of a tissue vessel, when disposed therein. The skilled person will immediately appreciate that the book shaped canister can be configured such that one or more channels can be located exclusively on either the cover side or the back side, or on complementary sides, so long as the book-shaped canister accommodates the structure of each port on the tissue vessel, when the canister is in a closed position.

The book-shaped canister is advantageously constructed from material capable of withstanding negative pressure in its interior chamber, without imploding, or compromising the canister structure. The book-shaped canister is also constructed so as to withstand any positive pressure generated from filling of a tissue vessel disposed therein with material. The book-shaped canister is thus desirably rigid and manufactured from a material such as polycarbonate, acrylic, ABS, ethylene vinyl acetate or styrene-butadiene copolymers (SBC) or the like. In preferred embodiments, the canister is manufactured from medical grade materials. The interior chamber of the book-shaped canister is desirably contoured in a shape complementary to a tissue vessel to be disposed therein. In embodiments wherein the tissue vessel is a flexible, collapsible bag, as described elsewhere herein, the interior chamber of the book-shaped canister is desirably contoured to prevent over-filling of the interior vessel chamber of the flexible, collapsible bag, as well as structural compromise of the flexible, collapsible bag.

FIG. 3C depicts a book-shaped canister in a closed position. In the embodiment shown in FIG. 3C, the cover and back sides are locked in a closed position via clasps. The skilled person will readily appreciate that various other locking mechanisms, e.g., clamps, and the like, could readily be used in the embodiments disclosed herein, to secure the cover and back sides in a closed position.

In some embodiments, the book-shaped canister is configured to house a heating element that heats, cools or adjusts the temperature of material in a tissue vessel within the canister. In some embodiments the heating element is integral to the canister chamber, e.g., is fixably attached to the back or cover side. In some embodiments, the heating element is removable.

In some embodiments, the back side of the book-shaped canister can include an equilibration port, that provides access to the interior chamber of the canister when in a closed position, and that is configured for attachment to a vacuum source. In some embodiments, the tissue vessel includes a suction port that is attached to the vacuum source. Accordingly, in some embodiments, the vacuum source can be connected, e.g., via conduits or other connection devices to the equilibration port of the canister and to a suction port of the tissue vessel. When the tissue inlet port of the tissue vessel is connected to a vacuum assisted tissue harvesting instrument, vacuum can be applied, and tissue will be drawn into the internal vessel chamber, while the pressure in the interior chamber of the canister and the interior vessel chamber are substantially the same.

### Methods

The embodiments disclosed herein advantageously allow for the direct harvest of tissue or other material into a processing device, e.g., tissue processing devices manufactured from flexible materials, as described herein above. Accordingly, the embodiments provided herein relate to the use of the devices described above for the harvest of tissue. Also provided are methods of harvesting tissue.

Some embodiments include steps of providing a tissue transfer device including a canister and a tissue vessel, and providing a vacuum source. The canister of the tissue transfer device can have an interior chamber, and is capable of withstanding negative pressure. The canister includes a body and a lid, and at least a first port and a second port that each has an interior end within the interior chamber of the canister. The exterior end of the first port is attached to a tissue harvesting instrument, such as a cannula. In some embodiments, the method includes the step of attaching the tissue harvesting instrument to the exterior of the first port, e.g., via a conduit or the like. The exterior end of the second port is attached to the vacuum source. In some embodiments, the methods include the step of attaching the vacuum source to the exterior end of the second port, e.g., via a conduit. The tissue vessel is disposed in the interior chamber of the canister. The tissue vessel includes a tissue inlet port attached to the interior end of the first port of the canister, and which provides access to the interior vessel chamber of the tissue vessel, i.e., a sterile fluid/tissue pathway to the interior vessel chamber. The tissue vessel also includes one or more venting ports that provides access into the interior vessel chamber (i.e., will allow the passage of material in an out of the interior vessel chamber). The vacuum source is actuated, and a vacuum is generated in the interior chamber of the canister and within the interior vessel chamber. Tissue is vacuumed through the vacuum-assisted tissue harvesting instrument, into the interior vessel chamber via the tissue inlet port.

Other embodiments provide steps of providing a tissue transfer device including a tissue canister and a tissue vessel, and providing a vacuum source. The canister includes a body and a lid, and at least a first (tissue), second (vacuum) and third (equalizing) port that each has an interior end within the interior chamber of the canister. The exterior end of the first port is attached to a tissue harvesting instrument, such as a cannula. In some embodiments, the method includes the step of attaching the tissue harvesting instrument to the exterior of the first port, e.g., via a conduit or the like. The second and third ports are coupled to a vacuum manifold that via a second conduit and a third conduit, coupled to the second (vacuum) and third (equalizing) port, respectively, on the canister. The second and third conduits are both coupled (e.g., via a Y-connector to a single conduit, or other configuration), to the vacuum source. A tissue vessel that includes at least a tissue inlet port and a suction port is disposed in the interior chamber of the canister. The tissue inlet port is attached to the interior end of the first (tissue) port of the tissue canister, e.g., either directly or via a sterile connecting device. The suction port is attached to the second (vacuum) port of the canister. The vacuum source is actuated, and a vacuum is generated in the interior chamber of the vessel and within the interior vessel chamber. Tissue is vacuumed through the vacuum-assisted tissue harvesting instrument into the interior vessel via the tissue inlet port.

### Kits

Provided herein are kits for the transfer of tissue to a tissue processing device that utilize negative pressure to facilitate transfer. In some embodiments, the kit includes a lid that is configured to releasably engage a canister body as described herein above. In some embodiments, the lid includes at least a first and a second port. When engaged with the canister body, the lid and canister body form an interior chamber of the canister. The first and second ports each have an exterior end located on the exterior of the canister, and an interior end located on the interior chamber, wherein exterior end of the first port is configured for attachment to a first conduit providing fluid/tissue pathway to a cannula for vacuum assisted tissue harvest, and the exterior of the second port configured for attachment to a second conduit providing a pathway to a vacuum source. The kits can further include the first and second conduits, as described herein above, configured to provide a functionally closed, sterile, fluid/tissue pathway from the exterior end of the first port of the canister lid to a tissue harvesting instrument (e.g., a cannula) and from the second port to the vacuum source. The kits can also further include a cannula for vacuum assisted tissue harvest. In some embodiments, the kit includes the vacuum source. In some embodiments, the kit includes a tissue vessel configured to be housed within the interior chamber of the canister, and that has a tissue inlet port and one or more venting ports that provide access to an interior chamber of the tissue vessel, as described herein above. In some embodiments, the kits provided herein include the canister lid and the tissue vessel. In some embodiments, the kits provided herein include the canister lid, the tissue vessel, and the canister body. In some embodiments, the kits provided herein include the canister lid, the tissue vessel, the canister body and the first and second conduit. In some embodiments, the kits provided herein include the lid, the canister body, the first and second conduits, and the vacuum source.

In other embodiments, the kit includes a lid that is configured to releasably engage a canister body as described herein above. In some embodiments, the lid includes at least a first, second port, and third port. When engaged with the canister body, the lid and canister body form an interior chamber of the canister. The first, second, and third ports each have an exterior end located on the exterior of the canister, and an interior end located on the interior chamber. The exterior end of the first port is configured for attachment to a first conduit providing fluid/tissue pathway to a cannula for vacuum assisted tissue harvest, and the exterior of the second port configured for attachment to a second conduit providing a pathway to a vacuum source. The exterior end of the third port is configured for attachment to a third conduit providing a pathway to a vacuum source. The second and third conduits can be connected to a Y-connector, which connects the second and third conduits to a fourth conduit, which provides a pathway to the vacuum source. The kits can further include the first, second and third (and fourth), conduits, as described herein above, configured to provide a functionally closed, sterile, fluid/tissue pathway from the exterior end of the first port of the canister lid to a tissue harvesting instrument (e.g., a cannula) and from the second and third ports to the vacuum source. The kits can also further include a cannula for vacuum assisted tissue harvest. In some embodiments, the kit includes the vacuum source. In some embodiments, the kit includes a tissue vessel configured to be housed within the interior chamber of the canister, and that has a tissue inlet port and a suction port that provide for the sterile ingress and egress of material in to an out of the interior chamber of the tissue vessel. In some embodiments, the kits provided herein include the canister lid and the tissue vessel. In some embodiments, the kits provided herein include the canister lid, the tissue vessel, and the canister body. In some embodiments, the kits provided herein include the canister lid, the tissue vessel, the canister body and the first, second, and third (and fourth) conduits. In some embodiments, the kits provide one or more valves that can function to block ingress and egress of material through one or more of the first, second, or third conduits, respectively. In some embodiments, the kits provided herein include the canister lid, the canister body, the first and second conduits, and the vacuum source.

### Positive Pressure-Based Tissue Transfer

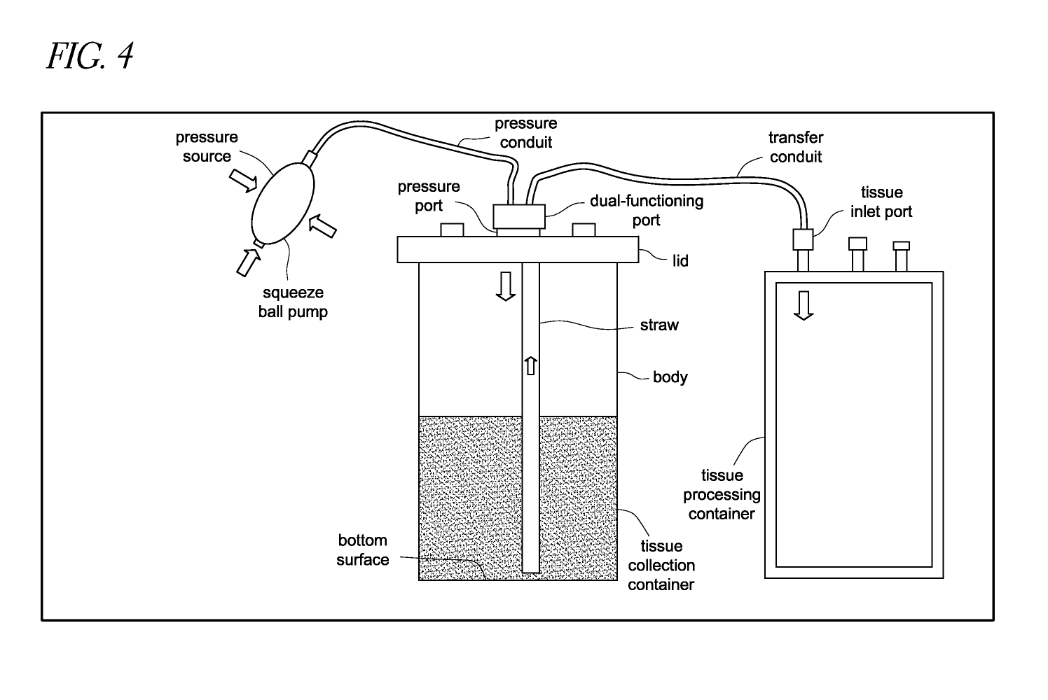

Some embodiments provided herein relate to methods and devices for the transfer of material, e.g., harvested tissue, or the like, from a tissue collection container to a tissue processing container. As explained in further detail below, in order to transfer tissue from within the internal chamber of a tissue collection container, a positive pressure source can be used to force gas into the interior chamber, which in turn displaces the tissue held within the internal chamber through a straw or conduit within the tissue collection chamber that provides access into a tissue processing container. Various exemplary, non-limiting embodiments of devices and the methods of their use for the transfer of tissue from a tissue collection container into a tissue processing container are shown in FIGS. 4-8.

Turning to the embodiments shown in FIGS. 4-8, the tissue collection container can be any type of container suitable for holding tissue, and include a body and a lid. Various commercially available tissue collection containers with tissue collection container bodies useful in the embodiments disclosed herein include, but are not limited to, for example the HI-FLOW™ canister (Bemis Health Care, Sheboygan Falls, Wis.), MEDI-VAC® suction canisters (Cardinal Health, Dublin, Ohio), hydrophobic suction canisters available from Bemis Health Care (Sheboygan Falls, Wis.), SAFELINER® suction canisters (DeRoyal, Powell, Tenn.), CRYSTALLINE™ suction canisters (DeRoyal, Powell, Tenn.), BERKELEY® SAFETOUCH® suction canister (Gyrus ACMI, Southborough, Mass.), CRD™ suction canisters (Cardinal Health, Dublin Ohio), FLEX ADVANTAGE suction canisters (Cardinal Health, Dublin Ohio), GUARDIAN™ suction canisters (Cardinal Health, Dublin Ohio), RECEPTAL® suction canisters (Hospira, Lake Forest, Ill.), DOLPHIN® suction canisters (Gyrus ACMI, Southborough, Mass.), and the like. A lid as described herein below can thus be coupled with a commercially available canister body following a liposuction procedure, in order to transfer harvested tissue, e.g., to a tissue processing device or tissue processing container.

Accordingly, the tissue collection container includes a lid that can be attached, e.g., releasably attached to, a canister body as described above. For example, in some embodiments, the canister includes a flange (e.g., a flange that that forms a rim of the top of the container), that engages a complementary groove in the lid. In some embodiments, the lid can be removable. In some embodiments, the lid remains attached to the tissue collection container body, e.g., via a hinging mechanism or the like. The body and lid of the tissue collection container assemble together to form an interior collection chamber, wherein the lid defines the top end of the interior collection chamber and the base of the collection container defines the bottom end of the interior collection chamber. The lid can include at least two ports, i.e., a pressure port and a transfer port, that each provides access into the interior chamber of the tissue collection container, where harvested tissue is contained. Each of the ports has an interior side facing the interior chamber of the tissue collection chamber, and an exterior side on the exterior surface of the lid. A pressure port disposed on the lid provides sterile access into the interior chamber of the tissue collection container and for the sterile ingress and egress of material (e.g., gases and the like) to the interior chamber of the tissue collection chamber. In some embodiments, instead of two separate ports (e.g., the pressure port and transfer port), the lid can include a single, dual-function port, but which is configured for attachment of a pressure conduit and a transfer conduit on the exterior end of the port, and for the attachment of a straw or any other structure having an internal lumen (e.g., flexible tubing or the like) through which material (e.g., gases, liquids, solids) can flow, on the interior end, as discussed further below.

A pressure conduit is connected to the exterior side of the pressure port, or the exterior side of a dual-function port, on a first end. In some embodiments, the first end of pressure conduit provides a sterile connection to the exterior side of the pressure port or dual-function port. The pressure conduit can be attached directly to the exterior side of the pressure port or dual-function port, or it can be attached using any suitable sterile connection device. A second end of the pressure conduit is connected to a pressure source capable of generating a positive pressure in the interior chamber of the tissue collection chamber. The pressure conduit can be attached directly to the pressure source, or it can be attached using a suitable sterile connecting device. In some embodiments, the pressure conduit includes a pressure relief valve. Preferably, the pressure conduit includes a filter, e.g., with a pore size of 0.5 μm or less, (e.g., less than 0.2 μm), that ensures that any material (e.g., gases or the like) that pass through the pressure port into the interior chamber of the tissue collection container is sterile. In some embodiments, the pressure conduit can include a check valve. In some embodiments, pressure relief valve includes a filter, e.g., with a pore size of 0.0.2 μm or less, that ensures that any material (e.g., gases or the like) that pass through into the interior chamber of the tissue collection container is sterile.

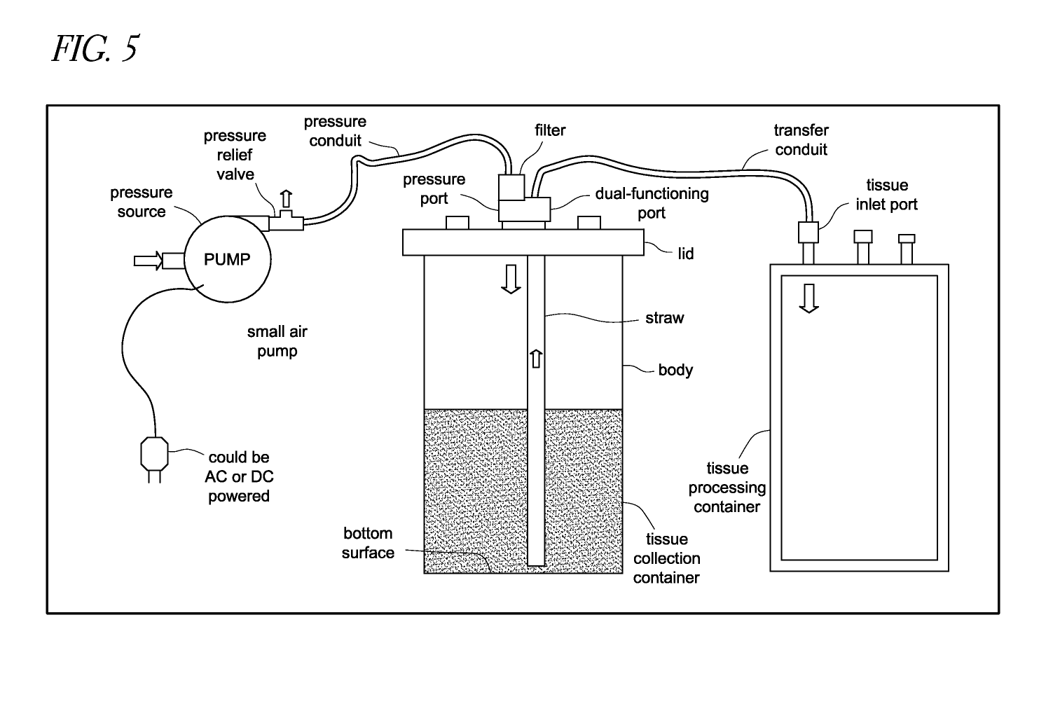

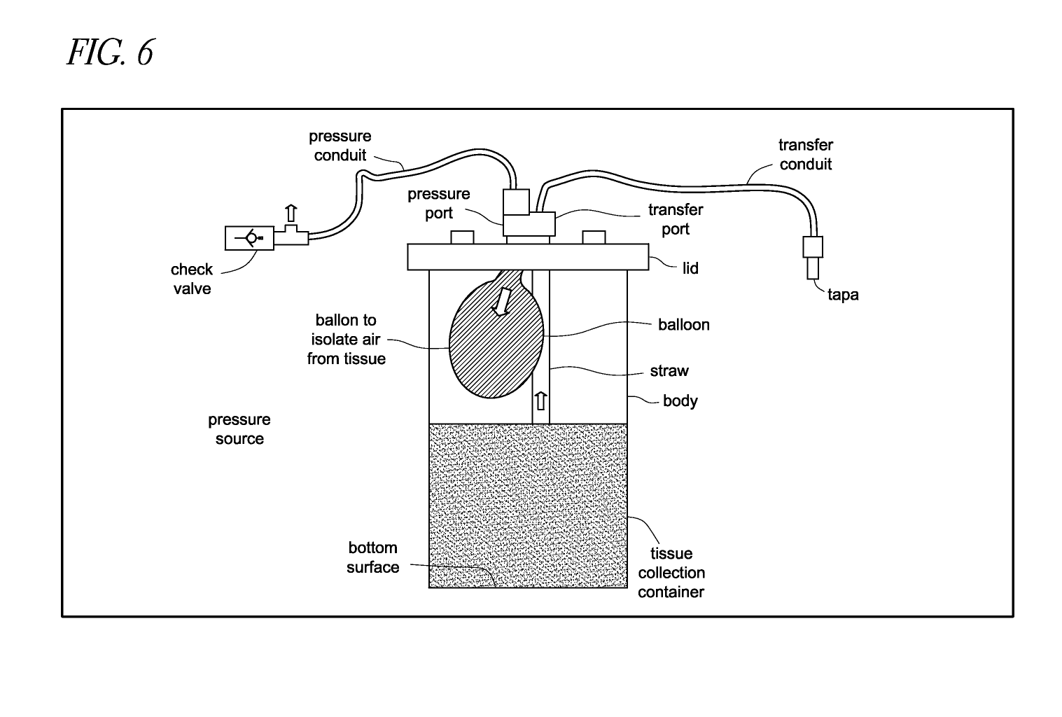

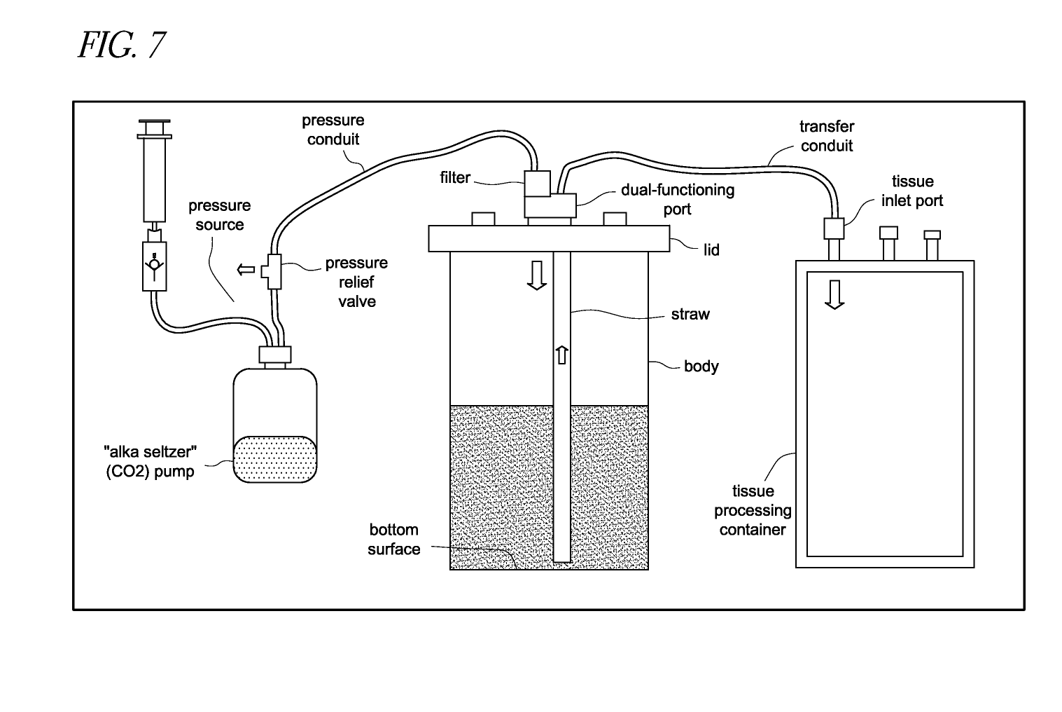

The pressure source can be manual, chemical, motorized, or the like. For example, the pressure source can include a hand pump, as illustrated in FIG. 4. Alternatively, the pressure source can be motorized, e.g., an air pump or the like, as illustrated in FIG. 5. In still other embodiments, the pressure source can be chemical as illustrated in FIG. 7. By way of example, in some embodiments, the chemical pressure source comprises a substance, such as alka seltzer, sodium bicarbonate, or the like, that produces gas when water or other reactant is added thereto. FIG. 6 depicts another exemplary embodiment of a tissue transfer system that utilizes positive pressure to transfer tissue from a tissue collection container to a tissue processing container under sterile conditions. In the embodiment shown in FIG. 6, a balloon (or other structure configured to receive gas or other material and to expand and/or contract upon being filled, e.g., an expandable body), is located within the interior chamber of the tissue collection container, and is connected to the interior of the pressure port, i.e., the end that is within the interior chamber of the tissue collection container. The pressure conduit is attached to a pressure source on a first end, and to the exterior end of the pressure port, i.e., the end that is on the exterior of the lid of the tissue collection container. The balloon or expandable body preferably isolates the material therein (e.g., gases or other material) from the contents, e.g., tissue, disposed within the interior chamber of the tissue collection container.

The interior end of the transfer port, or dual-function port, is connected to a top end of a straw or other structure having an internal lumen (e.g., flexible tubing or the like) that extends downwards from the transfer port into the body of the interior chamber of the tissue collection container, towards the bottom surface of the interior chamber. Preferably, the length of the straw is such that it extends to and rests against the bottom surface of the interior chamber (i.e., the bottom end or distal end of the straw or structure having an internal lumen rests against the bottom surface), however, in some embodiments, the bottom/distal end of the straw or structure having an internal lumen does not extend all the way to the bottom surface of the interior chamber. The straw (or other structure) has an interior lumen that allows for the passage of tissue therethrough. In some embodiments, the bottom end of the straw or structure having an internal lumen is angled to expose a larger area of the lumen and to thereby facilitate movement of tissue from the interior chamber into the straw. The straw can be rigid, or made of a flexible tubing or the like, configured for the sterile transfer of tissue, liquid, and other material therethrough. Preferably, the straw and other conduits are made from medical grade materials. The straw and other conduits are also preferably constructed of a transparent or translucent material to facilitate observation material passing therethough by the device operator.

The transfer conduit can include a first end that is configured for connection to the transfer port (or dual functioning port), and a second end that is configured for connection to the tissue processing container. The connections to the transfer port and to the tissue processing chamber can be direct, or can include a connecting device (e.g., a sterile connecting device). The tissue processing container has an interior chamber configured to receive tissue, and a tissue inlet port having an interior end and an exterior end, and that allows for the sterile access into the interior chamber of the tissue processing container. In some embodiments, the tissue processing container also includes one or more vent ports, auxiliary ports, drain ports, or the like, as described elsewhere herein.

The exterior end of the tissue inlet port is configured for sterile connection to the transfer conduit. The interior end of the tissue inlet port can include a conduit or straw or structure having an interior lumen which extends downwards into the interior chamber of the tissue processing container. In some embodiments, the tissue processing container is a flexible, collapsible bag comprising a mesh, wherein the mesh defines a first chamber and a second chamber of the interior chamber of the tissue processing container. The mesh can comprise a plurality of pores that pass liquids, tumescent fluids, red blood cells, and wash solutions and retain mature adipocytes, regenerative cells, stem cells, progenitor cells and connective tissue. The tissue processing container can also include a screen located within the second chamber, which creates a space between the mesh and the flexible, collapsible bag. The screen can be configured to wick tumescent fluids, red blood cells, and wash solutions from the first chamber into the second chamber of the interior chamber of the flexible, collapsible bag. The tissue processing container can include a tissue inlet port configured to allow the aseptic introduction of tissue into the first chamber of the flexible, collapsible bag; and one or more vent ports configured to provide sterile ingress and egress of material in and out of the tissue processing container. Exemplary tissue processing containers useful in the embodiments disclosed herein include, for example, flexible tissue processing containers such as those described in U.S. Patent Application Publication No. U.S. 2010/0279405, the disclosure regarding a flexible tissue container is herein incorporated by reference. Other non-limiting examples of tissue processing containers useful in the embodiments disclosed herein include, but are not limited to, those described in U.S. Pat. Nos. 7,390,484, 7,585,670, 7,687,059, 8,309,342, 8,440,440, U.S. Patent Application Publication No's. 2013/0164731, 2013/0012921, 2012/0164113, 2008/0014181. 2013/180952, 2013/0324966, 2010/0285521, 2013/0034524 International Patent Application Publication No. WO 2009/073724, WO 2013/030761, WO 2014/03969, WO 2013/106655, WO 2014/036094, and the like, each of which is herein incorporated by reference. In some embodiments, the tissue collection container and/or the tissue processing container has a capacity of at least 25 ml, 30 ml, 40 ml, 50 ml, 60 ml, 70 ml, 80 ml, 90 ml, 100 ml, 125 ml, 150 ml, 175 ml, 200 ml, 225 ml, 250 ml, 275 ml, 300 ml, 325 ml, 350 ml, 375 ml, 400 ml, 425 ml, 450 ml, 475 ml, 500 ml, 525 ml, 550 ml, 575 ml, 600 ml, 625 ml, 650 ml, 675 ml, 700 ml, 725 ml, 750 ml, 775 ml, 800 ml, 825 ml, 850 ml, 875 ml, 900 ml, 925 ml, 950 ml, 975 ml, 1 L, 1.25 L, 1.5 L, 1.75 L, 2 L, 2.25 L, 2.5 L, 2.75 L, 3 L, 3.25 L, 3.5 L, 3.75 L, 4 L, 4.25 L, 4.5 L, 4.75 L, 5 L, 5.25 L, 5.5 L, 5.75 L, 6 L, or greater, or any volume in between.

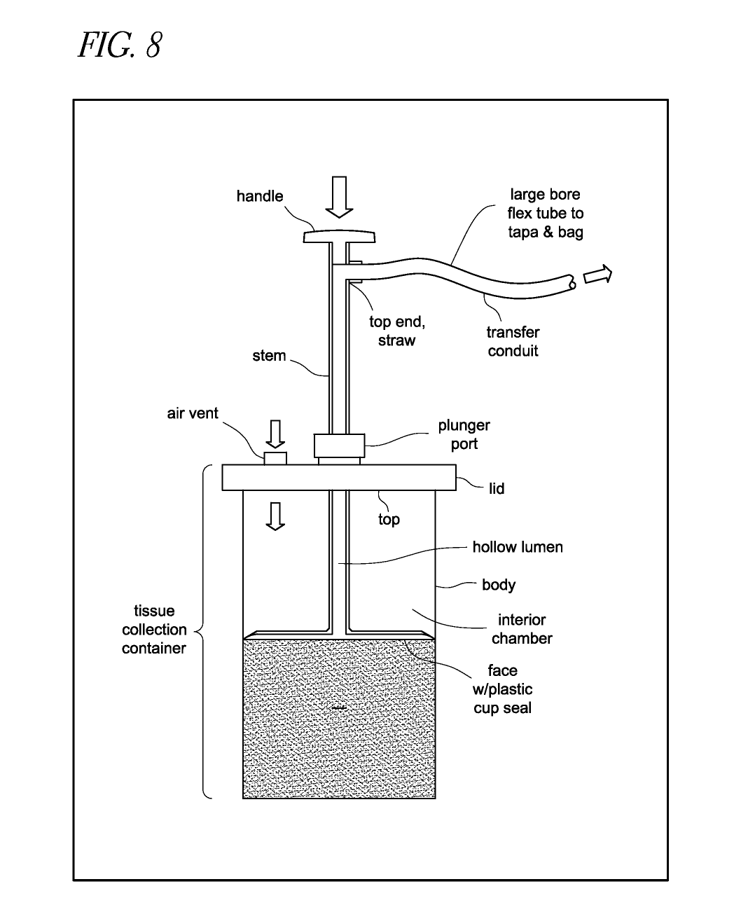

Turning to FIG. 8, shown is a tissue transfer device that utilizes a “French press” system to facilitate sterile transfer of tissue or other material from a collection container to another container, e.g., a tissue processing container. The embodiment shown in FIG. 8 includes a tissue collection container that has a body and a lid that is releasably attached to the vessel. The tissue collection container can be any type of container suitable for holding tissue, and include a body and a lid. As discussed above, various commercially available tissue collection bodies useful in the embodiments disclosed herein include, but are not limited to, for example the HI-FLOW™ canister (Bemis Health Care, Sheboygan Falls, Wis.), MEDI-VAC® suction canisters (Cardinal Health, Dublin, Ohio), hydrophobic suction canisters available from Bemis Health Care (Sheboygan Falls, Wis.), SAFELINER® suction canisters (DeRoyal, Powell, Tenn.), CRYSTALLINE™ suction canisters (DeRoyal, Powell, Tenn.), BERKELEY® SAFETOUCH® suction canister (Gyrus ACMI, Southborough, Mass.), CRD™ suction canisters (Cardinal Health, Dublin Ohio), FLEX ADVANTAGE suction canisters (Cardinal Health, Dublin Ohio), GUARDIAN™ suction canisters (Cardinal Health, Dublin Ohio), RECEPTAL® suction canisters (Hospira, Lake Forest, Ill.), DOLPHIN® suction canisters (Gyrus ACMI, Southborough, Mass.), and the like. The body of the tissue collection container can have a bottom and sidewalls. When attached to a lid, as described above, the body and the lid form an interior chamber of the tissue collection chamber.

The tissue transfer device of FIG. 8 also includes a lid. The lid can be coupled with a commercially available tissue collection container body following a liposuction procedure, in order to transfer harvested tissue, e.g., to a tissue processing device or tissue processing container. The lid can be attached, e.g., releasably attached to, the canister body as described above. For example, in some embodiments, the canister includes a flange (e.g., a flange that that forms a rim of the top of the container), that engages a complementary groove in the lid. In some embodiments, the lid can be removable. In some embodiments, the lid remains attached to the tissue collection container body, e.g., via a hinging mechanism or the like. The body and lid of the tissue collection container assemble together to form an interior collection chamber, wherein the lid defines the top end of the interior collection chamber and the base of the collection container defines the bottom end of the interior collection chamber. The lid can include a plunger port, which has two ends, i.e., an exterior end that faces the external environment and an interior end that faces and is located within the interior chamber of the tissue collection container. The plunger port houses a plunger. The plunger has a handle located on the exterior of the tissue collection container. The plunger also has a stem, which extends from the handle through the plunger port and into the interior chamber of the tissue collection container. The stem can form an air-tight seal with the plunger port, and is configured to slidably move through the port, such that the stem can be moved deeper into the interior chamber of the tissue collection chamber (i.e., toward the bottom of the interior chamber), or can be moved in the opposite direction (i.e., toward the top of the interior chamber), such that more of the stem is located on the exterior of the tissue collection container. The exterior surface of the stem can include structures such as a one-way ratchet, a rack, or a screw thread, that facilitates and/or directs movement of the stem deeper into (or out from) the interior chamber of the collection container. The plunger can include a face that forms a seal against the sidewalls of the interior chamber of the tissue collection container. In some embodiments, the face can be a flexible cup seal that surrounds a lumen. Regardless of the configuration of the plunger face, it comprises a lumen. The lumen can be a hollow structure, e.g., either rigid or flexible, that extends from the face of the plunger, and along the length of the stem (e.g., within the stem) towards the handle. The lumen can be connected to, or can be integral with, a transfer conduit located exterior to the tissue collection container. The transfer conduit can have an end that is configured for sterile connection (e.g., either directly or via a sterile connecting device) to a tissue inlet port of a tissue processing container, e.g., as described elsewhere herein, or to a cannula e.g., to facilitate transfer of tissue into the interior chamber of the tissue collection container.

In some embodiments, the lid can also include an air vent, e.g., that allows the passage of air in to an out of the interior chamber of the tissue collection container. In some embodiments, the air vent includes a filter, e.g., with a pore size of 5 μm or less, e.g., 4.0 μm, 3.0 μm, 2 μm, 1 μm, 0.9 μm, 0.8 μm, 0.7 μm, 0.6 μm, 0.5 μm, 0.4 μm, 0.3 μm, 0.2 μm, 0.1 μm, or less that ensures that any material (e.g., gases or the like) that pass through into the interior chamber of the tissue collection container is sterile. Preferably, the filter is less than 0.2 μm. In some embodiments, the air vent can be operably coupled to a vacuum source.

Provided herein are methods of using “French press” tissue transfer devices. In some embodiments, the method involves providing a tissue transfer body containing tissue therein. In some embodiments, the method includes attaching the lid to the body of the tissue collection container. In some embodiments, the lid is already attached to the tissue collection container. The method can include the step of applying force to the plunger, e.g., by pushing downwards on the handle of the plunger, such that the face of the plunger moves down the sidewalls of the body of the tissue collection container, towards the bottom. The face of the plunger eventually meets with the tissue disposed therein. As the plunger is moved deeper towards the bottom surface of the interior chamber, and once the face of the plunger comes into contact with the tissue (or other material) within the tissue collection chamber, the tissue is forced up through the lumen of the plunger face, and into the transfer conduit, and ultimately into the tissue processing container (e.g., through the inlet port of the tissue processing chamber).

### Methods

Some embodiments disclosed herein relate to the use of devices that utilize positive pressure to transfer tissue (or other material) from a tissue collection container to a tissue processing container.

In such embodiments, the methods can include the steps of providing a tissue transfer device that includes a tissue collection container. In some embodiments, the tissue collection container includes a body and a lid, wherein the body and lid fit together to form an interior collection chamber that houses tissue or other material. The body of the tissue collection container can be custom, or commercially available, e.g., as described herein above. In some embodiments, the tissue is in the interior chamber of the tissue collection container. In other embodiments, the methods include the step of transferring tissue into the interior chamber of the tissue collection chamber, e.g., using methods such vacuum assisted liposuction or the like (when the tissue is adipose tissue), or by manual means, and attaching the lid to the body of the tissue collection container after the tissue is transferred into the interior chamber.

The interior collection chamber has a top end, formed by the lid, and a bottom surface. The lid includes a pressure port that allows sterile ingress and egress of gas or material into and out of the interior collection chamber, and a pressure conduit that is sterilely connected to the pressure port, and that is configured for sterile connection to a pressure source capable of generating a positive pressure in the interior chamber of the collection container. The method can include the step of generating a positive pressure within the interior chamber of the tissue collection chamber. In some embodiments, generating a positive pressure comprises actuating a pump, e.g., a manual or automatic pump that is operably connected to the pressure conduit. In some embodiments, generation of a positive pressure comprises initiating a chemical reaction that releases gas that into the pressure conduit. The skilled artisan will readily appreciate that these examples are illustrative only, and that any method of generating positive pressure can be used in the embodiments disclosed herein. The positive pressure within the interior chamber of the tissue collection container eventually forces tissue up through a conduit located within the interior chamber of the tissue collection container, and which is connected to interior end of a transfer port on the tissue collection container lid. The tissue (or other material) moves through the transfer port into a transfer conduit that is connected to the exterior end of the transfer port on one (first) end, and to an inlet port on a tissue processing chamber on the other (second) end. In some embodiments, the method can further include the steps of removing the transfer conduit from the tissue processing container once the tissue is transferred thereto. In some embodiments, the method can further include the step of processing the tissue within the tissue processing container.

### Kits

Provided herein are tissue transfer kits that provide devices to facilitate the sterile transfer of tissue (or other materials) from a tissue collection container to a tissue processing container, e.g., using positive pressure to facilitate the transfer. In some embodiments, the kits include a lid that is configured for attachment to a body of a tissue collection container (e.g., a custom or commercially available tissue collection container as described elsewhere herein). In some embodiments, the kits do not include the body of the tissue collection container, whereas in some embodiments, the kits do include the body of the tissue collection container. In some embodiments, the kits include a tissue processing container. Accordingly, some embodiments provided herein relate to kits that include both a lid and a tissue processing container. Some embodiments provided herein relate to kits that include a lid, a tissue processing container, and a tissue collection container body as described above. In some embodiments, the kits include a transfer conduit as described herein above. In some embodiments, the kits include a pressure source, e.g., a pump, or structure and reagents for generation of gas pressure using a chemical reaction, or the like, as described elsewhere herein.

In some embodiments, the kits include instructions for using the tissue transfer devices described herein.

What is claimed is:

1. A tissue transfer device, comprising:
a canister having an interior chamber and an exterior, the canister being capable of withstanding negative pressure, wherein said canister comprises:
a body,
a lid,
at least a first port and a second port on the canister that each have an exterior end located on the exterior of the canister, and an interior end located on the interior chamber, wherein exterior end of the first port is configured for attachment to a first conduit providing fluid/tissue pathway to a cannula for vacuum assisted tissue harvest, and the exterior of the second port configured for attachment to a second conduit providing a pathway to a vacuum source;
a flexible tissue vessel disposed within the canister, said flexible tissue vessel comprising:
an interior vessel chamber;
a tissue inlet port that enables ingress and egress of material into and out of the interior vessel chamber, and which is configured for sterile connection to the interior end of the first port;
one or more venting ports that each enables ingress and egress of material into and out of the interior vessel chamber; and
a sterile connection between the interior end of the first port and the tissue inlet port of the flexible tissue vessel.

2. The tissue transfer device of claim 1, wherein the lid is removable.

3. The tissue transfer device of claim 1, wherein the lid is connected to the body via one or more hinges such that the lid provides access to the interior chamber of the canister when in an open position, and provides an air-tight seal with the body when in a closed position.

4. The tissue transfer device of claim 1, wherein the body of the canister comprises a bottom, a top, and sidewalls, and wherein the lid is configured to slide into an opening on the top of the canister, such that sliding the lid into the opening creates an air tight seal, and sliding the lid out of the opening provides access to the interior chamber of the canister.

5. The tissue transfer device of claim 1, wherein the first and/or second port is located on the lid.

6. The tissue transfer device of claim 1, wherein the first and/or second port is located on the body of the canister.

7. The tissue transfer device of claim 1, further comprising a vacuum source connected to the second conduit, to create a vacuum within the interior chamber of the canister, thereby facilitating suction of tissue through the cannula and into the interior vessel chamber.

8. The tissue transfer device of claim 1, further comprising a cannula for vacuum assisted tissue harvest, said cannula comprising a first end with a tissue inlet and a second end configured for sterile connection to the first conduit.

9. The tissue transfer device of claim 8, further comprising a sterile connector that connects the second end of the cannula to the first conduit.

10. The tissue transfer device of claim 1, wherein the tissue vessel comprises flexible, collapsible bag.

11. The tissue transfer device of claim 10, wherein the tissue vessel comprises:
a flexible, collapsible bag comprising a mesh, wherein the mesh defines a first chamber and a second chamber of the interior vessel chamber wherein the mesh comprises a plurality of pores that pass liquids, tumescent fluids, red blood cells, and wash solutions and retain mature adipocytes, regenerative cells, stem cells, progenitor cells and connective tissue;
a screen located within the second chamber of the flexible collapsible bag, wherein the screen creates a space between the mesh and the flexible, collapsible bag and said screen wicks tumescent fluids, red blood cells, and wash solutions from the first chamber into the second chamber of the flexible, collapsible bag;
the tissue inlet port configured to allow the aseptic introduction of tissue into the first chamber of the flexible, collapsible bag; and
the one or more vent ports configured to provide sterile ingress and egress of material in and out of the tissue vessel.

12. The tissue transfer device of claim 11, further comprising a drain port connected to the flexible, collapsible bag, wherein the drain port is configured for aseptic removal liquids, tumescent fluids, red blood cells, and wash solutions from the second chamber of the flexible, collapsible bag.

13. The tissue transfer device of claim 11, wherein the tissue vessel further comprises an auxiliary port configured to provide sterile ingress and egress of material in and out of the first chamber of the tissue vessel.

14. The tissue transfer device of claim 11, wherein the interior side of the first port of the canister is connected to the tissue inlet port of the flexible tissue vessel, and wherein the interior side of the second port of the canister is not coupled to the tissue vessel.

15. The tissue transfer device of claim 11, wherein the tissue inlet port comprises a straw extending into the first chamber of the tissue vessel, wherein the straw comprises an inner lumen configured to pass tissue therethrough.

16. The tissue transfer device of claim 1, wherein the canister body is a book-shaped body designed to accommodate an open and closed configuration, said book-shaped body comprising:
a cover side and a back side;
a first edge and a second edge; and
a bottom and a top, wherein the top comprises the canister lid,
wherein the first edge comprises a hinging means for movement of the body into an open position and a closed position by allowing movement of the cover side and back side relative to each other, wherein in the closed position, the interior chamber of the canister is sealed along the first and second edges, and the bottom and top to form an air-tight seal to the interior chamber; wherein when the book-shaped body is in an open position, the interior chamber of the canister is open to the external environment.

17. The tissue transfer device of claim 16, wherein the book-shaped canister is configured to house a heating element in the interior chamber that provides heat to the tissue vessel.

18. The tissue transfer device of claim 1, wherein when the vacuum source applies a vacuum through the second conduit, the vacuum created in the internal chamber of the canister outside of the tissue vessel is greater or equal to the vacuum in the interior vessel chamber.

19. A tissue transfer device, comprising:
a canister having an interior chamber and an exterior, the canister being capable of withstanding negative pressure, wherein said canister comprises:
a body,
a lid,
at least a first port, a second port, and a third port on the canister that each have an exterior end located on the exterior of the canister, and an interior end located on the interior chamber of the canister, wherein exterior end of the first port is configured for attachment to a first conduit providing fluid/tissue pathway to a cannula for vacuum assisted tissue harvest, the exterior of the second port configured for attachment to a second conduit providing sterile access to a vacuum source, and the exterior of the third port is configured for attachment to a third conduit that provides a sterile path to the second conduit;
a flexible tissue vessel disposed within the canister, said flexible tissue vessel comprising:
an interior vessel chamber;
a tissue inlet port that enables ingress and egress of material into and out of the interior vessel chamber, and which is configured for sterile connection to the interior end of the first port;
a suction port that enables ingress and egress of material in and out of the interior vessel chamber, and which is configured for sterile connection to the interior end of the second port.

20. The tissue transfer device of claim 19, wherein the third conduit is connected to the second conduit via a Y connector.

21. The tissue transfer device of claim 19, wherein the third port is located on the canister body.

22. The tissue transfer device of claim 19, wherein the third port is located on the canister lid.

23. The tissue transfer device of claim 19, wherein the third conduit comprises a valve configured to obstruct the passage of material into the second conduit in a closed position, and configured to allow the passage of material into the second conduit in an open position.

24. The tissue transfer device of claim 19, wherein the first conduit comprises a valve configured to obstruct the passage of material into the interior vessel chamber in a closed position, and configured to allow the passage of material into the interior vessel chamber in an open position.

25. The tissue transfer device of claim 19, wherein the second conduit comprises a valve configured to obstruct the passage of material from the internal chamber of the canister into the second conduit in a closed position, and configured to allow the passage of material from the internal chamber of the canister into the second conduit in an open position.

26. The tissue transfer device of claim 19, wherein the suction port of the tissue vessel is connected to interior end of the second port of the canister.

27. The tissue transfer device of claim 19, wherein the tissue vessel comprises:
a flexible, collapsible bag comprising a mesh, wherein the mesh defines a first chamber and a second chamber of the interior vessel chamber, wherein the mesh comprises a plurality of pores that pass liquids, tumescent fluids, red blood cells, and wash solutions and retain mature adipocytes, regenerative cells, stem cells, progenitor cells and connective tissue;
a screen located within the second chamber of the flexible collapsible bag, wherein the screen creates a space between the mesh and the flexible, collapsible bag and said screen wicks tumescent fluids, red blood cells, and wash solutions from the first chamber into the second chamber of the flexible, collapsible bag;
the tissue inlet port connected to the flexible, collapsible bag, wherein said inlet port is configured to allow the aseptic introduction of tissue into the first chamber of the flexible, collapsible bag;
the suction port connected to the flexible, collapsible bag configured to provide sterile ingress and egress of material in and out of the interior chamber of the tissue vessel; and
one or more venting ports that each enables ingress and egress of material into and out of the interior vessel chamber.

28. The tissue transfer device of claim 23, wherein the suction port is configured to provide sterile ingress and egress of material in an out of the second chamber of the tissue vessel.

29. The tissue transfer device of claim 27, wherein the third conduit is connected to the second conduit via a Y connector.

30. The tissue transfer device of claim 27, wherein the vacuum source comprises a waste trap configured to collect material directed from the drain port to the second conduit.

31. The tissue transfer device of claim 19, further comprising the vacuum source.

32. The tissue transfer device of claim 19, further comprising the cannula.

33. The tissue transfer device of claim 26, wherein the tissue inlet port comprises a straw extending into the first chamber of the tissue vessel, wherein the straw comprises an inner lumen configured to pass tissue therethrough.

34. The tissue transfer device of claim 19, wherein the canister body is a book-shaped body designed to accommodate an open and closed configuration, said book-shaped body comprising:
a cover side and a back side;
a first edge and a second edge; and
a bottom and a top, wherein the top comprises the canister lid,
wherein the first edge comprises a hinging means for movement of the body into an open position and a closed position by allowing movement of the cover side and back side relative to each other, wherein in the closed position, the interior chamber of the canister is sealed along the first and second edges, and the bottom and top to form an air-tight seal to the interior chamber; wherein when the book-shaped body is in an open position, the interior chamber of the canister is open to the external environment.

35. The tissue transfer device of claim 1, wherein when the vacuum source applies a vacuum through the second and third conduits, the vacuum created in the internal chamber of the canister outside of the tissue vessel is greater or equal to the vacuum in the interior vessel chamber.

36. A tissue transfer device for sterile transfer of tissue from a collection container to a processing container, comprising:
the tissue collection container, comprising:
a body;
a lid, wherein the body and the lid together form an interior collection chamber, wherein the interior collection chamber has a top end formed by the lid, and a bottom surface;
a pressure port to allow sterile ingress and egress of gas into and out of the interior collection chamber, said pressure port having an interior side within the interior collection chamber, and an exterior side;
a pressure conduit comprising a sterile connection to the pressure port, and configured for sterile connection to a pressure source capable of generating a positive pressure in the interior collection chamber;
a straw extending the interior side of the transfer port of the collection container into and towards the bottom surface of the interior chamber of the collection container, said straw having an interior lumen configured to pass tissue therethrough;
a transfer conduit that provides a sterile connection between the exterior side of the transfer port of the tissue collection container, and the tissue processing container, and which is configured to allow the transfer of tissue therethrough;
the tissue processing container, comprising:
an interior processing chamber configured to receive tissue therein;
a tissue inlet port having an exterior end configured for connection to the transfer conduit, and an interior end within the interior processing chamber.

37. The tissue transfer device of claim 36, wherein the processing container comprises:
a flexible, collapsible bag comprising a mesh, wherein the mesh defines a first chamber and a second chamber within said flexible, collapsible bag, wherein the mesh comprises a plurality of pores that pass liquids, tumescent fluids, red blood cells, and wash solutions and retain mature adipocytes, regenerative cells, stem cells, progenitor cells and connective tissue;
a screen located within the second chamber of the flexible collapsible bag, wherein the screen creates a space between the filter and the flexible, collapsible bag and said separator wicks tumescent fluids, red blood cells, and wash solutions from the first chamber into the second chamber of the flexible, collapsible bag;
the tissue inlet port connected to the flexible, collapsible bag, wherein said transfer port is configured to allow the aseptic introduction of tissue into the first chamber of the flexible, collapsible bag.

38. The tissue transfer device of claim 37, wherein the processing container further comprises a drain port connected to the flexible, collapsible bag, wherein the drain port is configured to aseptically remove liquids, tumescent fluids, red blood cells, and wash solutions from the second chamber of the flexible, collapsible bag.

39. The tissue transfer device of claim 36, wherein the pressure source is selected from the group consisting of a manual pump, an automatic pump, and a chemical pressure source.

40. The tissue transfer device of claim 36, wherein the pressure port comprises a filter.

41. The tissue transfer device of claim 36, wherein the pressure port comprises a balloon located on the interior side of the tissue processing container, said balloon configured to receive gas from the pressure source, and to displace gas present in the interior collection chamber.

42. The tissue transfer device of claim 36, wherein the pressure conduit comprises a pressure relief valve.

43. A tissue transfer device for sterile transfer of tissue from a tissue collection container to a tissue processing container, comprising:
the tissue collection container, said tissue collection container comprising:
a body;
a lid;
an interior chamber with a top, a bottom and sidewalls, defined by the body and lid of the tissue collection container, and an exterior;
a plunger port located in the lid, having an exterior end and an interior end facing the interior chamber of the collection container, wherein said port houses a plunger,
the plunger, the plunger comprising:
a handle located on the exterior of the canister;
a stem forming an air tight seal with the plunger port, said stem configured to slidably move down within the plunger port causing the stem to be moved deeper within the interior chamber of the collection container while maintaining the air-tight seal, said stem comprising:
a hollow lumen configured to allow the transfer of tissue therethrough;
an exterior surface;
a top end connected a transfer conduit providing a sterile pathway between the lumen and the tissue processing container or a cannula, said top end being connected to the handle;
a bottom end located within the interior chamber of the tissue collection container, said bottom end comprising
a face comprising a flexible cup seal surrounding the lumen, said flexible configured for moving up and down the sidewalls of the interior chamber;
an air vent located on the lid of the collection container, comprising a filter for sterile ingress and egress of gas in to and out of the interior chamber.

44. The tissue transfer device of 43, further comprising a vacuum source connected to the air vent.

45. The tissue transfer device of claim 43, wherein the exterior of the stem comprises a one-way ratchet, a rack, or a screw thread.

46. The tissue transfer device of claim 43, wherein the processing container comprises:
a flexible, collapsible bag comprising a mesh, wherein the mesh defines a first chamber and a second chamber within said flexible, collapsible bag, wherein the mesh comprises a plurality of pores that pass liquids, tumescent fluids, red blood cells, and wash solutions and retain mature adipocytes, regenerative cells, stem cells, progenitor cells and connective tissue;
a screen located within the second chamber of the flexible collapsible bag, wherein the screen creates a space between the filter and the flexible, collapsible bag and said separator wicks tumescent fluids, red blood cells, and wash solutions from the first chamber into the second chamber of the flexible, collapsible bag;
the transfer port connected to the flexible, collapsible bag, wherein said transfer port is configured to allow the aseptic introduction of tissue into the first chamber of the flexible, collapsible bag.

47. A method of harvesting a tissue directly into a tissue vessel for processing, comprising:
providing the tissue transfer device of claim 1, wherein the tissue inlet port of the tissue vessel is connected to the interior end of the first port;
providing a vacuum source connected to the second conduit;
providing a cannula for vacuum assisted tissue harvest attached to the first conduit;
generating a vacuum within the interior chamber of the canister via the vacuum source; and
vacuuming the tissue through the cannula, first conduit, and tissue inlet port into the interior chamber of the tissue vessel.

48. The method of claim 47, wherein the tissue is selected from the group consisting of adipose tissue, bone marrow, placenta, skin, eschar tissue, endometrial tissue, adult muscle, corneal stroma, dental pulp, Wharton's jelly, amniotic fluid, and umbilical cord.

49. A method of harvesting a tissue directly into a tissue vessel for processing, comprising:
providing the tissue transfer device of claim 19, wherein the tissue inlet port of the tissue vessel is connected to the interior end of the first port;
providing a vacuum source connected to the second conduit;
providing a cannula for vacuum assisted tissue harvest attached to the first conduit;
generating a vacuum within the interior chamber of the canister via the vacuum source; and
vacuuming the tissue through the cannula, first conduit, and tissue inlet port into the interior chamber of the tissue vessel.

50. The method of claim 47, wherein the tissue is selected from the group consisting of adipose tissue, bone marrow, placenta, skin, eschar tissue, endometrial tissue, adult muscle, corneal stroma, dental pulp, Wharton's jelly, amniotic fluid, and umbilical cord.

51. The method of claim 49,
wherein the tissue comprises adipose tissue,
wherein the first conduit comprises a valve configured to obstruct the passage of material into the interior vessel chamber in a closed position, and configured to allow the passage of material into the inter vessel chamber in an open position; and
wherein the third conduit comprises a valve configured to obstruct the passage of material into the second conduit in a close d position, and configured to allow the passage of material into the first conduit in an open position;
wherein the tissue vessel comprises:
a flexible, collapsible bag comprising a mesh, wherein the mesh defines a first chamber and a second chamber of the interior vessel chamber, wherein the mesh comprises a plurality of pores that pass liquids, tumescent fluids, red blood cells, and wash solutions and retain mature adipocytes, regenerative cells, stem cells, progenitor cells and connective tissue;
a screen located within the second chamber of the flexible collapsible bag, wherein the screen creates a space between the mesh and the flexible, collapsible bag and said screen wicks tumescent fluids, red blood cells, and wash solutions from the first chamber into the second chamber of the flexible, collapsible bag;
the tissue inlet port connected to the flexible, collapsible bag, wherein said inlet port is configured to allow the aseptic introduction of tissue into the first chamber of the flexible, collapsible bag;
the suction port connected to the flexible, collapsible bag configured to provide sterile ingress and egress of material in and out of the interior chamber of the tissue vessel; [location of suction port in bag is located not specified here] and
one or more venting ports that each enables ingress and egress of material into and out of the interior vessel chamber,
wherein the valves in the first, second and third conduits are in an open position during the harvesting step,
said method further comprising removing liquids, tumescent fluids, red blood cells, and wash solutions from the second chamber of the tissue vessel, following said harvesting step, said removing step comprising:
closing the valves in the first and third conduits; and
generating a vacuum within the second chamber of the interior vessel chamber via the vacuum source, thereby removing liquids, tumescent fluids, red blood cells, and wash solutions from the second chamber of the tissue vessel.
